(19) United States
(12) Patent Application Publication
(10) Pub. No.: US 2007/0196928 A1
(43) Pub. Date: Aug. 23, 2007

(54) Analytical Pretreatment Column

(76) Inventors: Kazuki Nakanishi, Kyoto (JP); Akiko Shinoda, Tokyo (JP); Haruhiko Ikeda, Kawasaki-shi (JP); Hiroshi Uchida, Yokohama-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 10/594,521

(22) PCT Filed: Mar. 30, 2005
(86) PCT No.: PCT/JP05/06719

§ 371 Date: Sep. 28, 2006

Related U.S. Application Data
(60) Provisional application No. 60/562,250, filed on Apr. 15, 2004.

(30) Foreign Application Priority Data
Mar. 31, 2004 (JP) 2004-104590
Dec. 15, 2004 (JP) 2004-363323

(51) Int. Cl. B01D 15/08; G01N 33/00

(52) U.S. Cl. 436/86; 210/656

(57) ABSTRACT

The analytical pretreatment column of the present invention is filled with at least the inorganic type filler having a number average diameter consisting of double pore structure having both a through pore with a number average diameter of the range of 0.5 to 25 micrometers, and meso pore with number average diameter of the range of 2 to 50 nm.

### TECHNICAL FIELD

The present invention relates to an analytical pretreatment column and a process for dispensing a target substance, a process for purifying a sample, and a process for recovering a target substance, in particular to an analytical pretreatment column for rapidly dispensing organic substances which are present in low levels in the environment such as in water and in the atmosphere, a process for producing an inorganic type filler for the analytical pretreatment column, a process for dispensing a target substance, an environmental purifying technology to which the above techniques are applied, or a technology of recovering minor constituents.

Priority is claimed on Japanese Patent Application No. 2004-104590, filed Mar. 31, 2004, U.S. Provisional Application No. 60/562,250, filed Apr. 15, 2004, and Japanese Patent Application No. 2004-363323, filed Dec. 15, 2004, the contents of which are incorporated herein by reference.

### BACKGROUND ART

Environmental pollution has become a problem worldwide. For example, by dissolving into sea water, evaporation, and rain, agricultural chemicals emitted from near the equator move to both poles little by little, and are destroying the ecosystem of the poles. Moreover, air pollution in China causes acid rain around the Sea of Japan coast. On the other hand, water-soluble organic compounds also move through water, such as river water, sea water, and groundwater. Moreover, although toxic substances, such as arsenic compounds currently used, for example for the chemical weapons, etc., have been buried and processed in the earth with the chemical weapon concerned, a possibility that these toxic substances will move through water is pointed out. Furthermore, although a shotgun may be used for animal extermination, lead may be contained in the bullet, and when the animal which is shot by the shotgun escapes, as a result, a high-concentration lead is emitted into the environment, and the lead moves through water or a high level animal. That is, not only organic and inorganic substances but any kinds of substance may also diffuse. In the case in which such a chemical substance is analyzed, in the sample, impurities which affect analysis are often contained in the usual analysis sample in many cases, in particular, in the sample which was extracted from the environment, many impurities (coexisting substance) exists at very high levels. Therefore, in order to conduct the analysis exactly, a pretreatment is necessary to reduce the content of the impurity (coexisting substance).

By the way, in order to reduce various impurities (coexisting substance), an extracting process and a purifying process are necessary, and hence there is a problem that these processes take a long time. Moreover, a part of the chemical substance which is the target for analysis may be removed in error through these pretreatment processes, and hence a method which can remove only impurities selectively is sought. Furthermore, a method in which an adsorbent which has a selective adsorbing property to the target substance of the analysis is used to adsorb the target substance thereto, and then the target substance is released by a solvent having a high extracting power, is also proposed. However, in this method, in particular, when the concentration of the target substance of the analysis is low, there is a problem that a large number of samples are required, and the processing takes a long time.

Here, as the chemical substance which is the target of analysis, for example, POPs (Persistent Organic Pollutants) may be exemplified. It is defined that POPs are organic substances which are harmful, difficult to decompose in nature, and are easily biologically concentrated in the food chain, and in addition, which is easily diffused by way of air or water and which has a bad influence on the health of the environment or a human beings even in the areas far from the place where they were produced, used or, discarded.

The POPs are regarded as problematic for the following reasons. In toxic substances, a highly volatile substance evaporates to be diffused into the air, whereas a low volatility substance does not gasify and tends to stop at the surface of the earth. However, POPs have a midrange volatility, in which evaporation and condensation take place comparatively easily. Therefore, POPs once gasify, moves through the air, and condense again to return to the surface of the earth. While repeating the cycle of this gasification and condensation, POPs are diffused to overall the earth. Furthermore, since most POPs are a chlorinated organic compounds, POPs are liposoluble and are biologically concentrated easily.

On the other hand, a water-soluble organic compound moves via water, such as river water, sea water, and groundwater. Furthermore, the water-soluble organic compound converts into the other compounds by way of excretion of human beings or livestock, etc., or metabolism in the living body, and then it moves.

For this reason, not only a conventional method such as chasing the causal relationship between factory effluents and drinking water, for example, monitoring of the mass transfer worldwide which includes a biological concentration is also becoming necessary with respect to numbers of chemical substances which remain in water, earth, or atmospheric air, or which are newly emitted to water, the ground, or the air. That is, monitoring which focused not only on the viewpoint of toxicity but also on the endocrine disruptor action is required about these hazardous property compounds, and development of various kinds of monitoring methods is advanced from two sides, such as (i) a long term sampling method for covering change by life activities, and (ii) a quick sampling method of the minor constituent.

Moreover, since PCB (polychlorinated biphenyl) which is one of the typical POPs has a stable chemical, physical, and useful characteristics, PCB had many uses. However, although manufacturing PCB was stopped in 1972 because of the toxicity thereof and a duty of storage thereof was imposed, PCB has been used until expiration at that time. Now, a serious PCB processing has started according to the law, and it is concerned about the indoor environmental pollution by breakage and disclosure of PCB content sealant or a fluorescent light stabilizer in the PCB processing institution within the indoor environment of the demolition areas, such as a transformer and a capacitor, or a storage place, and a building. Therefore, the necessity for the PCB measurement in the environment is increasing.

In a non-patent document 1 (The Ministry of Environment, Environmental Management Bureau administration division countermeasures-against-indoor dioxin and the “air environmental-research manual in connection with dioxin of Section 3 measurement analysis method of the dioxin in the environmental atmosphere)], [online], August, Heisei 13, the Ministry of Environment, and [September 8, Heisei 14 search], Internet <URL:http://www.env.go.jp/air/osen/manual/index.html>, the measurement manual of the dioxin which is POPs in the environmental atmosphere, and coplanar PCB is disclosed.

The measuring method disclosed in the non-patent document 1 is a so-called a high volume air sampler method. In the high volume air sampler method, an apparatus in which a sample sampling tools (quartz fiber filter paper and polyurethane form) are disposed to a high volume air sampler, for example, a product of SHIBATA Scientific Technology Ltd. model No.: HV-500F, HV-1000F (for dioxin) is used, and the high volume air sampler method is conducted by flowing the air thereto at a flow rate of 700 L/min. for 24 hours continuously, or 100 L/min for seven days continuously to be sampled therewith, such that the total amount of drawn atmospheric air becomes about 1,000 m³. However, this method has many problems; for example, it necessitates 24 hours or seven days for sampling, in addition, the apparatuses used therein are expensive and complicated, and in the case in which plural measurements are performed simultaneously at plural points, or plural substances including the other POPs are measured simultaneously, plural apparatuses are necessary.

Meanwhile, the non-patent document 2 (written by Takeshi NAKANO and six others “The Analytical Method of POPs in Ambient Air”, 10^{th} symposium on Environmental Chemistry Program and Abstract 10th environmental chemistry debate lecture summaries, May 23, Heisei 13, and p. 472-473) discloses, as a method for monitoring POPs using a comparatively simple apparatus, a low volume air sampler method (PS Air-Low-Vol. method) using a solid phase extracting column (brand name “Sep-Pak” (TR) PS Air, produced by Waters Extraction Corporation)

This method is a method of connecting the above solid phase extracting column (brand name “Sep-Pak” (TR) PS Air, produced by Waters Extraction Corporation) to a low volume air sampler (for example, air sampling pump, SP204-20L, manufactured by GL Sciences Inc.), and extracting the air by flowing it into this column at a flow rate of 2 to 5 L/min. for 24 hours.

Moreover, for example, in the non-patent document 3 (written by Takeshi Nakano and three others “Monitoring Method of PCB in Indoor Air” 10th Symposium on Environmental Chemistry Program and Absracts, May 23, Heisei 13, and p. 582-583) it is described that this method is also useful as a monitoring method of PCB in the indoor atmospheric air.

However, in the PS AirPS Air-Low-Vol method which is proposed by the monitoring technique of PCB in the above indoor atmospheric air, it is specified that extracting about 4 m³ of the indoor atmospheric air should be conducted for 24 hours, and hence the air flow rate in a solid phase extraction column becomes about 3 L/min. Thus, in the above method, the air flow rate is low, and hence there is a problem that the sampling takes a long time.

That is, there are no disclosures which indicate the method being capable of sampling toxic substances such as POPs in a liquid or gas, quickly, using simple equipment.

This PS AirPS Air-Low-Vol method is a solid phase sampling process using a column, and this kind of solid phase sampling process has come to be used frequently in recent years. Although a liquid absorbing method had been conventionally used for extracting a sample from a gas, whereas a liquid-liquid extracting process had been conventionally used for extracting a sample from a liquid, however, there is a problem that operation of these methods are complicated and these methods necessitate time, experience and a large amount of solvent be used. On the other hand, in the solid phase extracting method work, the operation can be easily performed and it takes a short time; moreover, the method necessitates a small amount of solvent be used. Therefore, when many samples must be processed in a short period of time, the solid phase extracting method is very advantageous, and it is easy to conduct the method automatically.

What lies behind the solid phase extracting method spreading quickly in recent years is that porous particles with sufficient absorbing and discharging performance are developed and that such a porous particle has come to be provided to the market as an adsorbent for solid phase extracting by plural makers. For example, as is disclosed in the patent document 1 (Japanese Unexamined Patent Application, First Publication No. S 59-147606 and the patent document 2 (Japanese Unexamined Patent Application, First Publication No. H4-334546), as for the filler used in the solid phase extracting, a silica gel or a chemical bond type silica gel in which the surface of a silica gel is chemically modified is employed as an inorganic type base material, whereas, as an organic type base material, a synthetic polymer which is represented by a copolymer styrene-divinyl benzene and a synthetic polymer of which the surface is chemically modified is employed.

By the way, in order to adsorb efficiently the persistent organic pollutants in extremely small quantity which exist in the air of an overlarge, using a disk or a column which is filled with the above adsorbent, it is preferred to reduce the particle diameter of the adsorbent particle to be as small as possible (for example, brand name GL-Pak PLS series by a GL Sciences Inc. or the Varian, Inc brand name of BOND ELUUTO series™ etc., in which these solid phase extract on column is filled with an adsorbent having a particle diameter of about 40 to 300 micrometers), or to increase the filling density of the adsorbent particles to reduce the gap among the adsorbent particles to be as small as possible (Those filled in a plus type cartridge. For example, brand name Sep-Pak™ PS Air, produced by Waters Corporation). However, if it is done so, the pressure which is applied at the time of ventilation will increase, and hence the flow rate cannot be increased, such that it takes a long time to flow a large amount of the air.

On the other hand, a high performance liquid chromatography (which is referred to as HPLC, hereafter) is a technique separating a mixture depending on the difference in the moving speed of each substance in the liquid flow by using a silica gel, polymer gel, etc., as a filler. In order to improve the performance of the HPLC, high resolution and high-speed separation are studied. It is preferred to reduce the diameter of a particle of the filler, in order to attain high resolution, and to reduce dead volume between filler particles. However, there is a limit in the equipment by increasing the pressure which is loaded to the column, and the difficulty in filling, and hence, as for many of filler particles, about 5-micrometer particle diameter serves as a limit as a compromise point of high resolution and high-speed separation in the present condition. Although the filler of small particle diameter, such as 1.5 micrometers and 2 micrometers, is also produced, because of a high back pressure, the length of a column should be reduced, or the flow rate of mobile phase must be decreased, and hence it is hard to say that true highly efficiently has been attained.

Then, as what breaks the limit of the above fine particulate filling type HPLC columns, silica gel of a double porepore structure having both a through pore of micrometer size and a meso pore of nanometer size was developed. The double porepore structure silica gel can control independently the diameter of through pore, and the diameter of the meso pore, and back pressure can be reduced by enlarging the through pore. For example, as is disclosed in the patent document 3 (Japanese Unexamined Patent Application, First Publication No. 2002-362918) and the patent document 4 (Japanese Unexamined Patent Application, First Publication No. 2003-075420), coexistence of a high efficiently resolution and a high-speed separation is attained by shaping the silica gel into a rod form and applying it to HPLC (one of column size is a cylindrical silica gel of one lump).

However, a trial of making the double pore structure silica gel selectively adsorb a small amount of a target substance mentioned above, or, conversely, a trial of removing only a small amount of the target substance out of the system without adsorbing it, was not yet performed.

Moreover, each above silica gel is used in a rod form, and hence there is a problem that such a rod form lacks mechanical strength. If the crushed double pore structure silica gel can be used, it can be thought that it leads to an improved productivity. Furthermore, the double pore silica gel having a rod form developed hitherto has a size of the maximum in about 10 micrometers as the size of the through pore. As is clear from the fact that the most of the base material for collecting gas has a particle diameter of about 500 micrometers, in order to flow gas through a through pore, it is not sufficient to have a size of 10 micrometers.

Hitherto, for example, as is disclosed in the patent document 5 (Japanese Unexamined Patent Application, First Publication No. H11-268923), as a trial of crushing a silica gel to produce a powdery silica, an example of crushing a wet gel of silica while passing it through a nylon net is known. Moreover, for example, as is disclosed in the patent document 6 (Japanese Unexamined Patent Application, First Publication No. H05-23565), a method for obtaining a granular silica gel by injecting a silica sol into an organic solvent through a polymer membrane having through holes of which diameter is uniform to form emulsion particles of the sol in the organic solvent, thereafter the emulsion particles are gelated, to obtain granular silica gel, is known. However, no examples where the granular silica gel was manufactured through the above method in manufacturing a double pore silica gel are reported hitherto.

### DISCLOSURE OF INVENTION

Although the inventors of the present invention have thoroughly researched to attain an analytical pretreatment column which can suppress the pressure which is applied when flowing a liquid or a gas, relatively low, while maintaining a high adsorbing ability and a high desorbing ability of a silica, they judged that it was very difficult to attain the purpose, as far as a powdery filler having a small pore size is filled into a column as it is, alternatively, a powdery filler having a small pore size is squeezed into a column with decompressing it such that the gap among the powder is reduced, and using such a column.

The present invention was made in view of the above situation, and an object of the invention is to provide an analytical pretreatment column which can dissolve the problems of the conventional technology, a process for dispensing a target substance, a process for purifying a sample, and a process for recovering a target substance, using the analytical pretreatment column.

That is, another object of the present invention is to provide a process which can purify the target substance quickly, easily, at a low cost, and a high reproducibility, and which can shorten the time required for performing a pretreatment of the sample which is analyzed, while maintaining a high recovery percentage.

In addition, still another object of the present invention is to provide an analytical pretreatment column which enables a pretreatment easily, at a low cost, and a high reproducibility, and which can shorten the time required for performing a pretreatment of the sample which is analyzed, while maintaining a high recovery percentage.

In addition, a further still another object of the present invention is to provide a process for producing a powdery inorganic type filler which consists of a double pore structure having both through pores and meso pores.

The present invention provides the following. Namely,

(1) An analytical pretreatment column characterized by being filled with at least an inorganic type filler comprising a double pore structure having both a through pore with a number average diameter of not less than 0.5 micrometers to not higher than 25 micrometers, and a meso pore with a number average diameter of not less than 2 nm to not higher than 50 nm.

(2) An analytical pretreatment column as set forth in (1), wherein the percentage of the pore volume of the through pore which occupies the volume of the inorganic type filler is ranging from not less than 30% to not higher than 90%.

(3) An analytical pretreatment column as set forth in (1) or (2), wherein the inorganic type filler has a coating of an organic compound thereon.

(4) An analytical pretreatment column as set forth in (1) to (3), wherein the inorganic type filler consists of a silica as a main component.

(5) An analytical pretreatment column as set forth in (1) to (4), wherein the inorganic type filler has a granular shape, a number average diameter of not less than 10 micrometers to not more than 1000 micrometers, and the specific surface area according to the BET method of not less than 50 m²/g.

(6) A process for producing an inorganic type filler, comprising:

hydrolyzing and gelating alkoxysilane in a solution containing a template compound to form a silica gel, grinding the silica gel, and then treating the silica gel with a basic solution, or, treating the silica gel with a basic solution, and then grinding the silica gel, and, calcining the silica gel.
   (7) A process for producing an inorganic type filler as set forth in (6), wherein the grinding the silica gel is performed by using a device having a porous plate with a plural penetrating holes which squeezes the silica gel into the penetration holes from one surface of the plate to grind the silica gel.
   (8) A process for producing an inorganic type filler as set forth in (6) or (7), wherein the template compound is any one of polyethylene oxide, poly oxy ethylene alkyl ether, and poly acrylic acid.
   (9) An analytical pretreatment column as set forth in (1) to (4), wherein the inorganic type filler is one produced by the process as set forth in (6) to (8).
   (10) A process for dispensing a dispense target substance, comprising:
flowing an analyzed sample containing a dispense target substance and a coexisting substance into the analytical pretreatment column as set forth in (1) to (5) or (9), thereby making the dispense target substance be adsorbed to the inorganic type filler, and
flowing an eluting solvent into the analytical pretreatment column, thereby debouching the dispense target substance from the analytical pretreatment column.
   (11) A process for dispensing a dispense target substance, comprising:
flowing an analyzed sample containing a dispense target substance and a coexisting substance into the analytical pretreatment column as set forth in (1) to (5) or (9), thereby making the dispense target substance be adsorbed to the inorganic type filler and debauching the dispense target substance from the analytical pretreatment column.
   (12) A process for dispensing a dispense target substance as set forth in (10) or (11), wherein the dispense target substance is one or more selected from the group consisting of dioxin, polychlorinated biphenyl, agricultural chemicals, endocrine disruptor, heavy metal, and protein.
   (13) A process for purifying a sample, comprising: flowing a sample containing a removal target substance and a coexisting substance into the analytical pretreatment column as set forth in (1) to (5) or (9), thereby removing the removal target substance from the sample.
   (14) A process for recovering a recovery target substance, comprising: flowing a sample containing a recovery target substance and a coexisting substance into the analytical pretreatment column as set forth in (1) to (5) or (9), thereby recovering the recovery target substance from the sample.

The inventors of the present invention have been thoroughly researching for achieving an analytical pretreatment column which can suppress the back pressure which is applied to when flowing a liquid or a gas into the column comparatively low, while maintaining a high adsorbing capability and a high desorbing capability of a silica, and as a result, it is turned out that both objects can be attained by introducing pores (through pore) which can separate quickly into a silica base material, in addition to pores (meso pore) which can perform high-performance analysis. That is, with use of the above analytical pretreatment column, it is possible to suppress the back pressure which is applied to the column upon flowing a liquid or a gas into the column, comparatively low, while maintaining a high adsorption and desorption capability of a silica, thereby increasing the flowing rate of a liquid or a gas, it becomes possible to perform a pretreatment of a pretreatment target substance, quickly.

Moreover, it is known well that when performing analysis of a Liquid sample and a pretreatment using a column in which a common particle-like porous silica gel is filled, it is hard to remove air bubbles from the column, once air bubbles enter into the column, thereby making it difficult to use the surface of the silica gel effectively, and it leads to a deterioration of concentrating performance. However, in the inorganic type filler having a double pore according to the present invention, since there are through pores therein, it is thought that, even if air bubbles enter into the column, the air bubbles will be pushed out again by the solvent, and hence the entire surface of the silica can be utilized, and as a result, the concentrating performance will not deteriorate.

This means that it is not necessary to be careful particularly such that air bubbles might not enter into the column when performing a pretreatment, and hence it is thought to be useful from the viewpoints of reproducibility and of convenience.

According to the present invention, which can provide an analytical pretreatment column which can realize simple and quick dispersion of a small amount of a analysis target substance from an analyzed sample which is subjected to an analysis, while maintaining easiness, an excellent reproducibility and a high recovering percentage.

### BRIEF DESCRIPTION OF THE DRAWINGS

FIG. 1 is a cross-sectional schematic view showing the analytical pretreatment column which is one embodiment of the present invention.

FIG. 2 is a schematic view explaining an example of the concentrating method of an organic substance using the analytical pretreatment column which is one embodiment of the present invention.

FIG. 3 is a schematic view explaining another example of the concentrating method of an organic substance using the analytical pretreatment column which is one embodiment of the present invention.

FIG. 4 is graph which shows the relation between the accumulated pore volume of the inorganic type filler of Examples 2 and 3, and a pore diameter.

FIG. 5 is graph which shows the accumulated pore volume of the inorganic type filler of Examples 2 and 3, and the relation of the differentiation value and pore diameter.

FIG. 6 is graph which shows the relation between the differentiation value of the accumulated pore volume of the inorganic type filler of Examples 2 and 3, and a pore diameter in the range with a pore diameter of 1 to 100 nm.

### EXPLANATION OF A SYMBOL

1—Analytical pretreatment column,
2—Inorganic type filler,
2a—Filler layer,
3—Column container (reservoir)

### BEST MODE FOR CARRYING OUT THE INVENTION

Hereafter, the best mode for carrying out the present invention will be explained in detail.

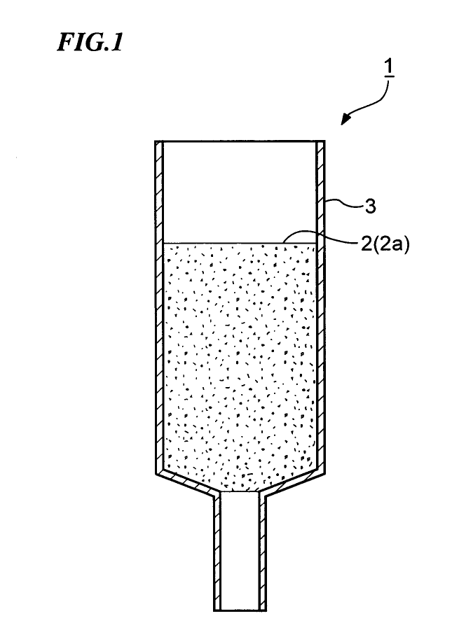

As shown in FIG. 1, an analytical pretreatment column 1 which is an example of this embodiment is constituted by filling up a syringe type container (reservoir) which is a column container 3, with an inorganic type filler 2 as a filler layer 2a, which consists of double pore structure having through pores with a number average diameter ranging of not less than 0.5 micrometer to not more than 25 micrometers, and meso pores with a number average diameter ranging of not less than 2 nm to not more than 50 nm. Moreover, the filter (illustration is omitted) is attached to the upper and lower sides of the filler layer 2a. The inorganic type filler 2 according to the present invention may be modified (coated) with an organic compound for controlling adsorbent of the surface, and may not be modified (coated) at all. Moreover, the column container 3 may be filled up with a mixture of the above inorganic type filler and a filler other than the above inorganic type filler 2, if needed, and it is also possible to fill the column container 3 with the inorganic type filler 2 and the other filler or base material so as to form a multilayer structure.

The shape of the column container 3 (reservoir) is not particularly limited. It may be a usual cylinder shape or may be a disk-shape. The size of the column container 3 can be a suitable size, corresponding to the amount of the analyzed substance. Usually, a size having a volume of 0.1 to 100 ml, preferably a size having a volume of 3 to 6 ml is suitable in respect of handling. Moreover, as a material of the column container 3 (reservoir), glass, a stainless steel, and a resin (for example, polypropylene, polyethylene) are preferred, and the material and the shape of the column container 3 is not particularly limited, as far as it is insoluble to a solvent which is used and the inorganic type filler 2 does not flow out of the column container 3 during the concentrating operation of a sample.

By flowing a sample such as a liquid or a gas into the analytical pretreatment column to make a target substance be adsorbed once, thereafter extracting the target substance, alternatively, by making a coexisting substance be adsorbed, thereby flowing out the target substance, it is possible to dispense or recover the target substance. In addition, purifying a sample is also possible.

Although the shape of the inorganic type filler 2 according to the present invention may be rod like (the filler serves as the size almost the same as one lump as the inside of a column container), bulk like (lump of about 1 mm to 5 cm), particle like, or a fiber like, a rod like shape or a particle like shape is preferred. In the case in which the shape of the inorganic type filler 2 is particle like, either irregular particles or spherical particles may be employed. When the shape is particle like, the number average diameter is preferably a ranging of not less than 10 micrometers to not more than 1000 micrometers, more preferably ranging of not less than 30 micrometer to not more than 700 micrometers, and the most preferred is a ranging of not less than 50 micrometers to not more than 500 micrometers.

If the average diameter is less than 10 micrometers, when the flow rate of the analyzed samples such as a liquid or a gas which flows into the column 1, is increased, the difference (pressure loss) of static pressure before and after the filler layer 2a which consists of the inorganic type filler 2 becomes large, and the analyzed sample cannot be passed at a high flow rate. Moreover, if the average diameter exceeds 1000 micrometers, since the adsorption efficiency of the target substance will deteriorate, it is not preferred. It should be noted that the average diameter of the inorganic type filler 2 is measured according to JIS Z8815 sieving test method general rules using the sieve for an examination provided in JIS Z 8801.

Moreover, the inorganic type filler 2 can also be used in combining those having the above shape. For example, it is also possible to use it, putting those having a shape of a particle or a fiber in the gap among the bulk-like fillers.

The specific surface area of the inorganic type filler 2 according to the present invention is preferably not less than 50 m²/g, and more preferably not less than 100 m²/g. Since the adsorption efficiency of the target substance will deteriorate if the specific surface area is less than 50 m²/g, it is not preferred. It should be noted that the specific surface area is measured by the BET method.

Moreover, the inorganic type filler 2 according to the present invention is preferably made of silica. Moreover, it is particularly preferred that the inorganic type filler 2 has a double pore structure, which is a pore structure consisting of through pores having a number average diameter ranging from not less than 0.5 μm to not more than 10 μm, and meso pores having a number average diameter ranging from not less than 2 nm to not more than 50 nm, each of which are connected to each other to be spread in three-dimensions. It should be noted that the through pore has a lot of penetration holes which penetrate inside the filler, and a meso pore has a lot of pore formed in the filler surface or the wall surface the through pore.

The number average diameter of the through pore is preferably ranging from not less than 0.5 μm to not more than 25 μm, more preferably ranging from 0.5 μm to not more than 10 μm, particularly preferably ranging from 0.5 μm to not more than 7 μm. If the number average diameter of the through pore is less than 0.5 μm, when the flow rate of the analyzed samples, such as a liquid, gas, etc., which flows into the column 1, is increased, since the difference (pressure loss) of static pressure before and after the filler layer 2a becomes large and the analyzed sample cannot be passed at a high flow rate, and hence it is not preferred. Moreover, if the number average diameter of the through pore is more than 25 μm, since the percentage of void of inorganic type filler 2 itself becomes large and it becomes impossible to maintain the physical intensity of the inorganic type filler 2, and hence it is not preferred.

The percentage of the pore volume of the through pore which occupies in the volume of the inorganic type filler is preferably ranging from not less than 30% to not more than 90%, more preferably ranging from not less than 40% to not more than 85%, and still more preferably ranging from not less than 50% to not more than 80%. If the percentage of the pore volume of the through pore is less than 30%, when the rate of the analyzed samples, such as a liquid, gas, etc., which flows into the column 1, is increased, since the difference (pressure loss) of static pressure before and after the filler layer 2a becomes large and the analyzed sample cannot be passed at a high flow rate, and hence it is not preferred. Moreover, if the percentage of the pore volume of the through pore is more than 90%, since the percentage of void of the inorganic type filler 2 itself becomes large and it becomes impossible to maintain the physical intensity of the inorganic type filler 2, and hence it is not preferred.

Moreover, the percentage of the pore volume of the through pore which occupies the volume of the inorganic type filler can be obtained from the product of the density of the inorganic type filler and the pore volume of the through pore per mass of the inorganic type filler.

The density of the inorganic type filler can be obtained by dividing the total volume of the inorganic type filler which includes both pore volume of the through pore and the pore volume of the meso pore by the mass of the inorganic type filler. Moreover, the pore volume of the through pore per the mass of the inorganic type filler can be obtained by the mercury pressing method.

It should be noted that as for a rod like inorganic type filler, the total volume which includes the volume of silica, the pore volume of the through pore and the pore volume of the meso pore can be comparatively easily obtained, and hence the calculation of the density can be performed with comparatively high accuracy, but as for a bulk like, a particle like, or fiber like inorganic type filler, calculation of the total volume is difficult, and as a result, it is often impossible to calculate the density of the inorganic type filler. In such a case, what is necessary is to make the adjustment conditions of gel the same, and an inorganic rod-like type filler is prepared, and the density of this inorganic rod-like type filler is used for calculating the percentage of the pore volume of the through pore.

Next, the number average diameter of the meso pore is preferably ranging from not less than 2 nm to not more than 50 nm, more preferably ranging from not less than 5 nm to not more than 40 nm, and particularly preferably ranging from not less than 8 nm to not more than 35 nm. If the number average diameter of the meso pore is less than 2 nm, the substance which is a target for adsorption cannot enter into the meso pore, and as a result, the substance becomes hard to be adsorbed, thereby decreasing the recovery percentage, and hence it is not preferred. Moreover, if the number average diameter is more than 50 nm, the substance becomes hard to be adsorbed sufficiently because of lack of surface area, and hence it is not preferred.

The inorganic type filler 2 according to the present invention can be produced, for example, by the following methods.

First, a water-soluble compound which serves as a template is added to an acidic solution, and is dissolved therein. Thereafter, alkoxysilane compounds such as tetra-methoxysilane and tetra-ethoxysilane, are added to hydrolyze this alkoxysilane compound. Thereafter, hydrolysis and polycondensation reactions are advanced further, and this solution is allowed to stand until solution gels and silica (silica gel) is formed. At this step, the diameter of the through pore is determined. That is, the diameter of the through pore can be determined by composition of starting solution, and controlling a rate of polymerization and controlling the grade of condensation of a water-soluble compound.

Next, the gelled silica is washed with water, and thereafter the gelled silica is treated with a basic solution. In this process, the dissolution and a re-deposit of a silica frame take place, and a meso pore is formed. Finally, the gel is washed with water, and then calcined at a high temperature to remove the water-soluble compound which becomes a template, thereby obtaining the inorganic type filler 2 according to the present invention.

In the present invention, as the above acidic solution, solutions such as one containing a concentration 0.01M to 11.0M of nitric acid, acetic acid, or the like can be used. Moreover, as a water-soluble compound which serves as a template, a polyethylene oxide, a polyoxy ethylene alkyl ether, a polyacrylic acid, having a molecular weight of about 100,000, etc., can be used. As the amount of addition of this water-soluble compound, for example, it can be considered to be ranging from 2 mass % to 15 mass %, to the above acidic solution. Moreover, aqueous urea, aqueous ammonia, etc., can be used as the basic solution for treating the gelled silica. Furthermore, as a calcining conditions for silica, it can be considered as 2 hours or the conditions which are calcined for 10 hours at the calcining temperature of 300° C., or at 600° C. Moreover, the inactive gas of calcining atmosphere, for example, nitrogen gas atmosphere etc., is preferred.

According to the process mentioned in the above, the inorganic type filler 2 according to the present invention can be obtained. The shape of the inorganic type filler 2 will become the shape which corresponds to the inner shape of the storage container of the silica at the time of calcination. For example, if the inner shape of the storage container is cylindrical, the inorganic type filler 2 having a cylindrical shape (a rod like shape) will be obtained. Moreover, an inorganic particle-like type filler 2 is obtained by further grinding the inorganic type filler 2 having a rod-like shape.

Moreover, an inorganic type filler having a particle-like shape can also be manufactured as follows.

That is, similar to the above mentioned producing method, after adding a water-soluble compound which serves as a template into an acidic solution and dissolving it therein, an alkoxysilane compound is added to be hydrolyzed, thereby forming a silica gel.

Next, the silica gel thus obtained is ground and then washed with water, thereafter the silica gel is treated with a basic solution. Alternatively, the silica gel obtained may be washed with water in advance, then treated with a basic solution, and thereafter ground. What is necessary is in short, the silica gel may be ground during after the silica gel is formed, and before the silica gel is calcined.

And after the ground silica gel is washed with water, the silica gel is calcined at a high temperature to remove the water-soluble compound which becomes a template, thereby obtaining a particle like inorganic type filler 2. Thus, grinding is performed before calcining, destruction and the blocking of pores caused by grinding processing can be reduced, and hence it is more preferred.

As a means for grinding a silica gel, the silica gel can be ground by squeezing the silica gel into penetration holes, from one surface of a porous plate material which has plural penetration holes. As the porous plate, for example, a metal net, a screening material, etc., may be exemplified. The pore size of the penetration holes is preferably ranging from 30 to 800 μm, more preferably ranging from 100 to 700 μm, and the most preferably ranging from 200 to 500 μm. If a pore size is not more than 500 μm, it is possible to control the diameter of a particle after calcinations in the proper range. Moreover, if the pore size is not less than 100 μm, it is possible to reduce the pressure upon squeezing the silica gel into the porous plate.

At this time, if carrier fluid is applied to the exit of the porous plate upon squeezing the silica gel thereinto from one surface of the porous plate, it is possible to grind the silica gel by forming micelle in the carrier fluid. As the carrier fluid, as far as it is the medium which is not uniformly mixed with the silica gel, any of fluid can be used without being limited. Preferably, a liquid having a low compatibility with water can be used. As a liquid having a low compatibility with water, for example, organic solvents, such as hexane, toluene, and octanol, can be used. If a surface-active agent is added to the carrier fluid used at this time, since the silica gel and the carrier fluid will be emulsified, the silica gel can be ground homogeneously. As a surface-active agent, the surface-active agent of a cationic type or a nonionic type can be used, and the surface-active agent of an alkyl sulfonic acid type and the poly oxy ethylene type can be used. Specifically, sodium dodecylbenzenesulfonate, polyethylene-glycols alkyl ether, etc., can be used.

It should be noted that gelling is not completed in a short time and there is width of time in a start and end of gelling to some extent. If it is going to be extruded out and fabricated after gelling is completed completely, when it is pushed out, pressure will be applied and extrusion will become difficult. Therefore, extruding and fabricating it before gelling completely is more preferred. Moreover, as the grinding method, the spray-drying method and the W/O emulsion method are known in addition to the above method, as is disclosed in Japanese Unexamined Patent Application, First Publication No. H07-069617, and it is also possible to perform the grinding using these methods.

It should be noted that as a method similar to the above-mentioned manufacture method it is indicated in detail by the following references 1 and 2, for example, and the inorganic type filler according to the present invention may be produced based on these references.

“Reference 1”

Norio Ishizuka and two others, “Monolithic Silica column for high-efficiency chromato graphic separation”, Hyomen, Vol. 39 and No. 8, p. 297-307, 2001.

“Reference 2”

Kazuki Nakanishi, “Pore Structure Control of Silica Gels Based on Phase Separation”, Journal of Porous Materials, 1997 and 4, 67-112

Next, although the obtained inorganic type filler may be used in the state of any of the shape of a rod, the shape of bulk, and the shape of a particle, it is preferred to coat the surface of the inorganic type filler with an organic compound if it is necessary to change the adsorbing characteristics thereof. Although, as the coating method, the monomer adsorption polymerizing method (how to polymerize a monomer on the inorganic type filler surface), a coupling reaction, etc., may be exemplified, as far as it is a compound which can react with the inorganic type filler, any compound can be employed without being limited in particular. As the organic compound which performs coupling reaction, chlorosilane compounds, which are used advantageously in liquid chromatography, are preferred. As a chlorosilane compound, dimethylchlorosilane, trimethylchlorosilane, aminomethyldimethylchlorosilane, dimethoxymethylchlorosilane, dimethylvinylchlorosilane, chloromethyltrimethylsilane, chloromethylmethoxydimethylsilane, chloromethyltrimethoxysilane, allyldimethylchlorosilane, dimethylpropylchlorosilane, 1-chloroethyltrimethylsilane, dimethylisopropylchlorosilane, methylphenylchlorosilane, dimethyloctadecylchlorosilane, tribenzylchlorosilane, dimethylphenylchlorosilane, triphenylchlorosilane, and diphenylmethylchlorosilane, etc., may be exemplified; however, it is not limited thereto.

Moreover, in addition to the above coupling reaction, when the inorganic type filler is silica, it is possible to react with the silanol portion thereof, and an organic metallic compound, a clathrate, protein, peptide, glycoprotein, sugar peptide, sugar, etc., may be exemplified.

The coupling reaction itself is a well-known reaction, and it is performed using a general method. For example, the method disclosed in the following reference 3 may be used. That is, pyridine and a silane coupling agent are added into toluene as a solvent in the absence of water, and it is made to react by heated reflux. A terminating reaction may be performed after washing and drying, if necessary.

“Reference 3”

Japanese Unexamined Patent Application, First Publication No. 2002-22721

In the analytical pretreatment column 1 of the present invention, it is also possible to use together the usual filler, for example, polystyrene beads, ODS, and an alumina bead, besides the inorganic type filler 2. That is, it is possible to use them by mixing the particles or fiber of a usual filler with the particles of the above inorganic type filler 2, or by packing them in layers into the column container.

As the adsorption target substance of the analytical pretreatment column of the present invention, the compound which is accumulated in people or livestock and causes hazardous conditions, the compound excreted in the urine of people or livestock and metabolites thereof or a compound useful for people or livestock, an environmental pollutant, a toxic substance, and a physiologically active substance may be exemplified. Specifically, the metabolism things, such as dioxin, polychlorinated biphenyl, agricultural chemicals, an endocrine disrupter, a petrochemical derivative, nutritional information and metabolites thereof, microbe products, toxin, and heavy metal, may be exemplified. As an example of these compounds, dioxin, dibenzofran, and polycyclic aromatic hydrocarbon (including PAHs and a benzo (a) pyrene), polychlorinated biphenyl, polybrominated biphenyl, DDT, chlorpyrifos, aldrin, dieldrin, endrin, chlordane, heptachlor, trichlorobenzene, tetrachlorobenzene, hexachlorobenzene (HCB), mirex, toxaphene (camphechlor), hexachloro cyclohexane (lindane (gamma-HCH) etc.), chlordecone (kepon), octachloro styrene (OCS), asulam, simazine, 1,4-dinoxane, nonylphenol, a surface-active agent, estrogens, androgens, the other hormone, polyphenols, antibiotic, antibacteria medicine, protein, peptide, lipid, sugars, nucleic acid related substance, vitamins, a neurotransmitter, a natural poison represented by a mycotoxin and marine toxin, arsenic, selenium, an metabolite thereof, etc., may be exemplified; however, it is not limited thereto in particular.

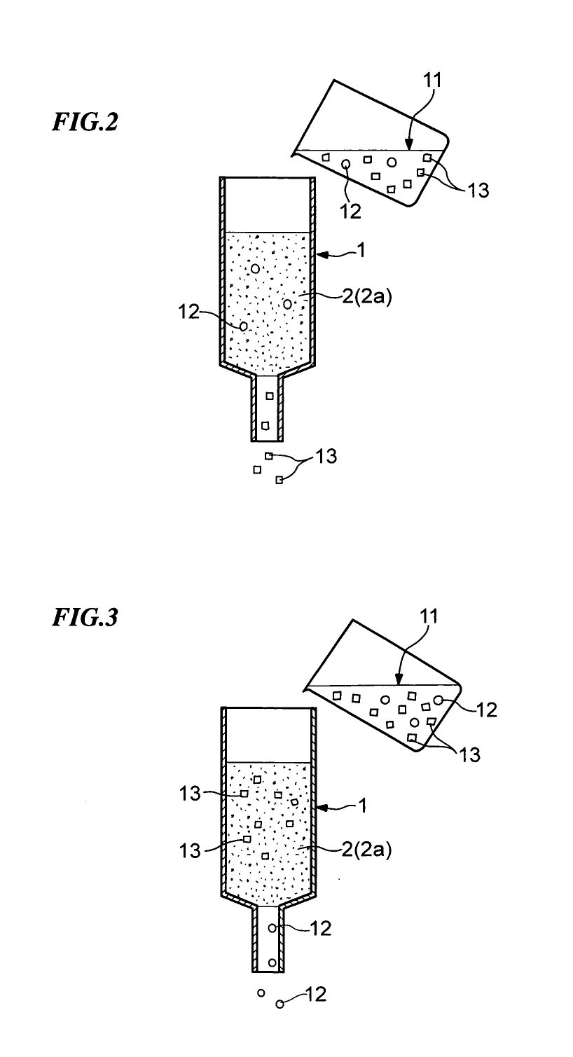

The analytical pretreatment column 1 of the present invention can be used as follows, for example. An example of the dispensing method of the target substance using the analytical pretreatment column 1 is shown in FIG. 2.

As shown in FIG. 2, the analytical pretreatment column 1 filled up with the inorganic type filler 2 is prepared, and a sample 11 is flowed down to the analytical pretreatment column 1. In this sample 11, a dispense target substance 12 and a coexisting substance 13 which is not the target of dispensing are contained. When the sample 11 is flowed down, the dispense target substance 12 is adsorbed by the inorganic type filler 2, whereas the coexisting substance 13 which is not the target of dispensing is flowed down as it is without being adsorbed. Thereafter, the eluant is flowed down to desorb the dispense target substance 12. Thus, the dispense target substance 12 contained in the sample 11 can be dispensed.

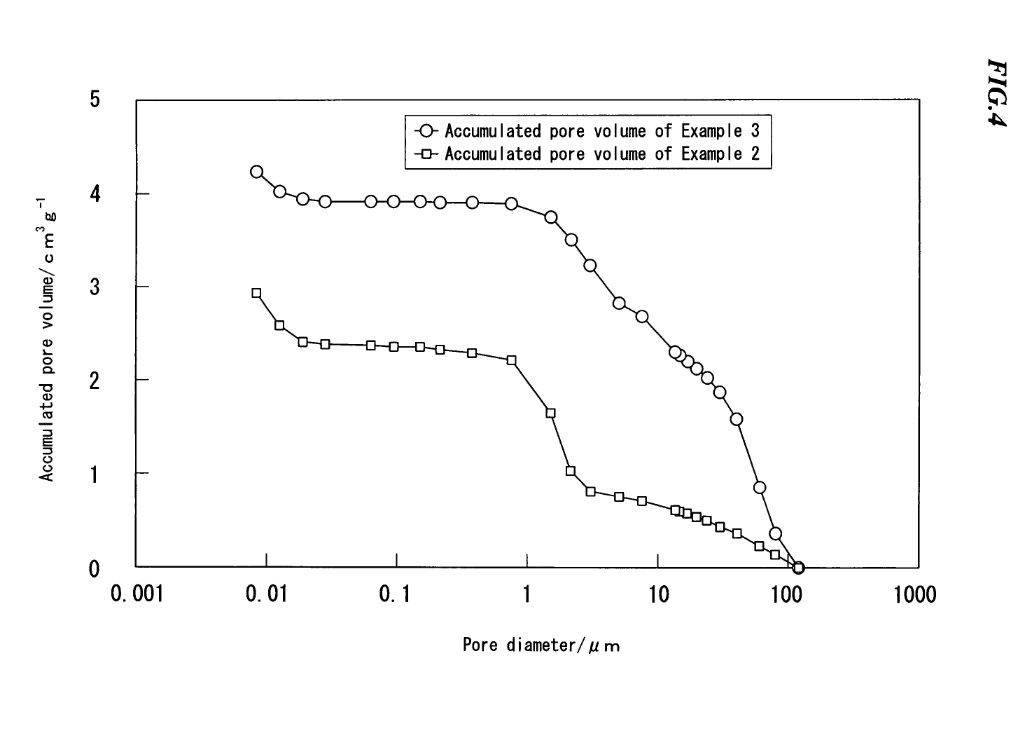

Moreover, another example of the dispensing method of the target substance using the analytical pretreatment column 1 is shown in FIG. 3.

As shown in FIG. 3, the analytical pretreatment column 1 filled up with the inorganic type filler 2 is prepared, and the sample 11 is flowed down to the analytical pretreatment column 1. When the sample 11 is flowed down, in the example of FIG. 3, the coexisting substance 13 which is not the target of dispensing is adsorbed by the inorganic type filler 2, whereas the dispense target substance 12 is flowed down as it is, without being adsorbed. While the coexisting substance 13 content in the solution flowed down being significantly reduced, most of the dispense target substance 12 flows out as it is. Thus, the dispense target substance 12 contained in the sample 11 can be dispensed.

In order to make the dispense target substance 12 be adsorbed by the inorganic type filler 2 or not to make it be adsorbed, it can be controlled by, for example, selecting the organic compound which coats the surface of the inorganic type filler 2, suitably.

As mentioned above, although the dispensing method of the target substance was explained, in the present invention, the above dispensing method is applicable to the method for removing the target substance from the sample, and the method for recovering the target substance as it is.

As mentioned in the above, according to the analytical pretreatment column 1 of this embodiment, the silica base material is equipped with the through pores which can perform a high speed isolation, in addition to the meso pores which can perform a high efficient analysis, and hence it is possible to suppress the back pressure which is applied to the column upon flowing a liquid or a gas into the column, comparatively low, while maintaining a high adsorption and desorption capability of a silica, thereby heightening the selectivity to the target substance, and shortening the pretreatment time.

Although, as a concrete use of the analytical pretreatment column of the present invention, a trace amount of substance selected from incinerator exhaust gas, various type of exhaust gas from factory, the environmental atmosphere such as the atmosphere collected over the trunk road, the indoor air, factory effluent, river water, lake water, the excrement from a human body and livestock, etc., and a pretreatment which needs advanced concentration may be exemplified, it is not limited thereto in particular.

### EXAMPLES

Hereinafter, although the present invention will be explained more concretely using ExampleExamples, the present invention is not limited by these ExampleExamples.

“Manufacturing of the Analytical Pretreatment Column of Example 1”

(Synthesis of Silica)

20 g of 1M nitric acid solution was prepared as an aqueous acid, and 1.2 g (molecular weight Mv=100,000, produced by Aldrich Co., Ltd.) of polyethylene oxide which serves as a template was added to the nitric acid solution to be dissolved therein. Next, after sorbitol (produced by Wako Pure Chemical Co., Ltd.) in an amount of 1.0 g was added to the solution to be dissolved, it was cooled under an ice bath. A tetra-ethoxysilane (produced by Shin-Etsu Chemical Co., Ltd.) in an amount of 14 ml as an alkoxysilane compound was added under an ice-cooling, while agitating violently. After agitating for 30 minutes under ice-cooling, 2 ml thereof was moved to a cylindrical container having an inner diameter of 12 mm, and was allowed to stand in a temperature controlled bath which was kept at 60° C. Tetra-ethoxysilane was hydrolyzed and gelled for about 2 hours after being allowed to stand, and became solid-like. This gel which was solidified cylindrically was allowed to stand in a pure water (ion exchanged water) for 8 hours to be washed with the pure water. Next, after the pure water was discharged, the gel was put into a 100 ml capacity autoclave, and 70 ml of 1.5M urea solution as a basic solution was added thereinto. Being allowed to stand for 2 hours, the mixture was heated to 110° C. for 3 hours, and then the gel was held for 5 hours to be aged. After-cooling down, the gel was taken out and it was washed by being allowed to stand in a pure water for 8 hours. After the pure water was discharged, the gel was moved to a crucible, and it was heated to 600° C. for 2 hours and sintered at 600° C. for 2 hours to decompose the polyethylene oxide (template) to be removed. Thus, a rod like silica gel having a double pore structure, i.e., a framework in which the meso pores frame and the through pores frame are tangled was obtained. The specific gravity of the thus obtained silica gel was 0.2 g/cm³.

Next, the above rod like silica gel in an amount of 1.6 g was refluxed with a 6 mol/L of hydrochloric acid in an amount of 50 mL for 5 hours to be washed. After the hydrochloric acid and the gel were filtrated, it was washed with a pure water until the filtrate became neutral. Thereafter, it was washed with acetone and dried at 130° C. for 4 hours to obtain silica gel which was washed and treated with a hydrochloric acid.

As to thus obtained silica gel, BET specific surface area, the number average diameter of meso pore, the number average diameter of through pore, and pore volume were measured. BET surface area was measured by the BET method using ASAP2000 produced by Micrometrics Co., Ltd.

The number average diameter of meso pore was calculated by obtaining the specific surface area and pore volume from the amount of adsorption of the nitrogen at the boiling point of liquid nitrogen (−196° C.), and computed the pore diameter based on the following formula (pore are regarded to be a cylindrical shape).
pore diameter=4×pore volume/specific surface area

Furthermore, the number average diameter and the pore volume of through pore were measured by the mercury pressing method using PORESIZER9320 produced by Micrometrics Co., Ltd.

Furthermore, the percentage of the pore volume of through pore which occupies in the volume of the inorganic type filler was calculated from the pore volume of through pore and the silica gel specific gravity.

In Table 1, the number average diameter of through pore, the number average diameter of meso pore, the BET specific surface area of silica gel, the pore volume of through pore, and the percentage of the pore volume of through pore in the through pore and the silica gel are shown.

Next, a silane coupling reaction was performed using the obtained silica gel, and the octadecyl group was introduced into the silica surface. Specifically, silica gel in an amount of 1.58 g which was washed and treated with a hydrochloric acid was dispersed into a dried toluene in an amount of 15 mL, and a dimethyl octadecyl chlorosilane (produced by Shin-Etsu Chemical Co., Ltd.) in an amount of 2.0 g and a pyridine (produced by Pure Chemistry Co., Ltd.) in an amount of 0.6 g were added, and the mixture was heated for 6 hours while being refluxed. Thereafter, the silica gel was washed with toluene in an amount of 5 mL, methanol in an amount of 50 mL, 20% water containing methanol in an amount of 50 mL, and chloroform in an amount of 20 mL, in this sequence. By performing a vacuum drying of the obtained silica gel at 130° C. for 4 hours, a rod-like silica gel chemically bounded octadecyl group was introduced was obtained. Thus, the inorganic type filler of Example 1 was produced.

(Preparation of the Analytical Pretreatment Column)

Next, the rod-like silica gel of Example 1 was inserted into a cylindrical column container (reservoir) having an inner diameter of 12 mm and an internal volume of 6 ml, of which bottom is equipped with a filter made of polyethylene, and then plugging from upper part thereof by another filter which is made of polyethylene and pushing it on with a stick, thereby preparing the analytical pretreatment column of Example 1.

It should be noted that an hardening epoxy resin was thinly applied onto the side surface of the rod like silica gel, then hardened the epoxy resin after being inserted into the column container, thereby sealing the slight gap between the silica gel and the inner surface of the column container.

“Preparation of the Analytical Pretreatment Column of Example 2”

The rod like inorganic type filler which was produced by the same way as in Example 1 was ground until the average diameter thereof became 80 μm to obtain a particle-like inorganic type filler. An analytical pretreatment column of Example 2 was prepared in the same way as in Example 1 with the exception of having filled up the reservoir with 200 mg of this inorganic type filler. However, since sealing with an epoxy resin is unnecessary in the case in which a particle-like filler is used, it is not performed. It is the same also in the following Examples and Comparative Examples.

Moreover, BET specific surface area, the number average diameter of meso pore, the number average diameter of through pore, and pore volume were measured as to the particle like inorganic type filler after being ground.

BET specific surface area and the number average diameter of meso pore were measured by the BET method using ASAP2000 produced by Micrometrics Co., Ltd. Moreover, the number average diameter and pore volume of through pore were measured by the mercury pressing method using the poreseemeter series 200 produced by CARLO ERBA Co.,Ltd.

Furthermore, the percentage of the pore volume of through pore which occupies in the volume of the inorganic type filler was calculated from the pore volume of through pore and the specific gravity of the inorganic type filler. Measurement results are shown in Table 1.

“Preparation of the Analytical Pretreatment Column of Example 3”

Similar to Example 1, 1.2 g of polyethylene oxide was added to 20 g of 1M nitric acid solution to be dissolved therein.

Next, after sorbitol in an amount of 1.0 g was added to the solution to be dissolved, it was cooled under an ice bath. A tetra-ethoxysilane in an amount of 14 ml was added under an ice-cooling, while agitating violently. After agitating for 30 minutes under ice-cooling, 2 ml thereof was moved to a cylindrical container having an inner diameter of 12 mm, and was allowed to stand in a temperature controlled bath which was kept at 60° C. Tetra-ethoxysilane was hydrolyzed and gelled for about 2 hours after being allowed to stand, and became solid-like.

Next, the gel which was solidified cylindrically was ground by extruding from a mesh of a stainless steel having an opening diameter of 300 μm regulated by JIS8801.

The ground gel was allowed to stand in a pure water for 8 hours to be washed with the pure water. Next, after the pure water was discharged, the gel was put into a 100 ml capacity autoclave, and 70 ml of 1.5M urea solution as a basic solution was added thereinto. Being allowed to stand for 2 hours, the mixture was heated to 110° C. for 3 hours, and then the gel was held for 5 hours to be aged. After-cooling down, the gel was taken out and it was washed by being allowed to stand in a pure water for 8 hours. After the pure water was discharged, the gel was moved in a crucible, and it was heated to 600° C. for 2 hours and sintered at 600° C. for 2 hours to decompose the polyethylene oxide (template) to be removed. Thus, a particle like inorganic type filler having a double pore structure, i.e. a framework in which the meso pores frame and the through pores frame are tangled was obtained.

Similar to Example 2, as to the thus obtained inorganic type filler, BET specific surface area, the number average diameter of meso pore, the number average diameter of through pore, and pore volume were measured, and the percentage of the pore volume of through pore which occupies the volume of the inorganic type filler was calculated.

Measurement results are shown in Table 1.

Next, the obtained inorganic type filler was classified to obtain a ground type inorganic type filler having an average diameter of 380 μm. The analytical pretreatment column of Example 3 was produced by the same way as in Example 1 with the exception of having filled up the reservoir with 300 mg of the inorganic type filler.

“Preparation of the Analytical Pretreatment Column of Comparative Example 1”

An analytical pretreatment column of Comparative Example 1 was prepared by the same way as in Example 1, with the exception of having filled up the reservoir with as a silica, Sep-Pak™ Vac C18 (the average pore diameter of 12.5 nm, the number average particle diameter of 55 μm to 105 μm) produced by Waters Co., Ltd.

“Preparation of the Analytical Pretreatment Column of Comparative Example 2”

An analytical pretreatment column of Comparative Example 2 was prepared by the same was as in Example 1 with the exception of having filled up the reservoir with a 300 mg of D-350-120A produced by Dohkai Chemical Industries Co., Ltd. (which has a pore diameter of 12 nm, and a number average particle diameter ranging from 250 μm to 500 μm) as a silica.

TABLE 1

| | | | | Pore | | |
|---|---|---|---|---|---|---|
| | Number Average | | BET | Volume | Percentage | |
| | diameter | | specific | of | of pore | |
| | Through | Meso | surface | through | volume of | |
| | Pore | Pore | area | pore | through pore | |
| | (μm) | (μm) | (m²/g) | (cm³/g) | (%) | Remarks |
| Example 1 | 2 | 15 | 150 | 3.5 | 70 | Rod like |
| Example 2 | 2 | 9 | 55 | 2.9 | 58 | Example 1 was |
| | | | | | | ground |
| Example 3 | 20 | 6 | 94 | 4.2 | 84 | Extruded |
| | | | | | | Through a mesh |

“Evaluation of Physical Properties of the Inorganic Type Filler of Examples 1-3”

As shown in Table 1, it turns out that the range of the number average diameter of through pore is 2 μm to not more than 25 μm, the number average diameter of meso pore is ranging from 6 nm to 15 nm, and each inorganic type filler of Examples 1-3 is an inorganic type filler according to the present invention.

Next, comparing Example 1 with Example 2, it turns out that as to the specific surface area, the pore volume of through pore, and the percentage of pore volume, Example 2 is inferior. This is because the inorganic type filler of Example 2 is one which is obtained by grinding the inorganic type filler of Example 1, and it is thought that a crack begins at the through pore upon grinding, and that the inside of the through pore until then is exposed to the surface of the ground particles, thereby hiding a part of the through pore.

Next, comparing Example 2 with Example 3, it turns out that as to the specific surface area, the pore volume of through pore, and the percentage of the pore volume, Example 3 is superior to. It is thought that this is because when producing the inorganic type filler of Example 3, the gel which contains the template is extruded through a mesh to be ground, and thereafter sintering the ground gel to form through pores, and hence there is no possibility of breaking the through pores to be decreased, upon grinding.

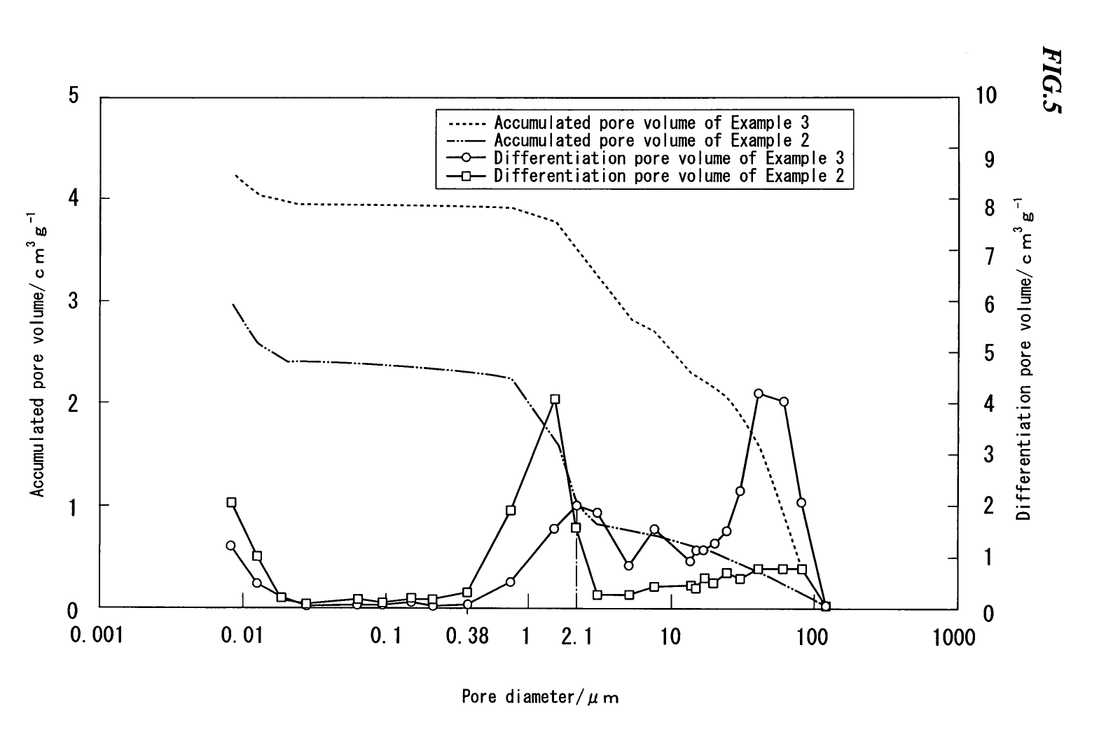

The correlations among the differentiation value of the pore diameter of Examples 2 and 3, the accumulated pore volume, and the accumulated pore volume are shown in FIGS. 4 to 6 in graphs, respectively. It should be noted that these graphs were prepared based on the measurement data of the number average diameter of meso pore by the BET method, and the number average diameter data and pore volume data of through pore by the mercury pressing method, and they serve basic data of the number average diameter, the percentage of pore volume, and pore volume on Table 1.

In the graph of FIG. 4, the accumulated pore volume is one which is obtained by accumulating the volume of all of pores formed in the inorganic type filler which includes the meso pores and the through pores. That is, FIG. 4 is a graph in which the vertical axis is set as accumulated pore volume, and the horizontal axis is set as the diameter of all of the pores containing meso pores and through pores.

As shown in FIG. 4, it turns out that the accumulated pore volume of Example 3 is higher than that of Example 2 in the range from 0.01 μm to 120 μm pore diameter. Thereby, in the Example 3, it turns out that the volume of the pores containing through pore and meso pore is higher than that of Example 2, and that it excels in the adsorption characteristic to the adsorption target substance.

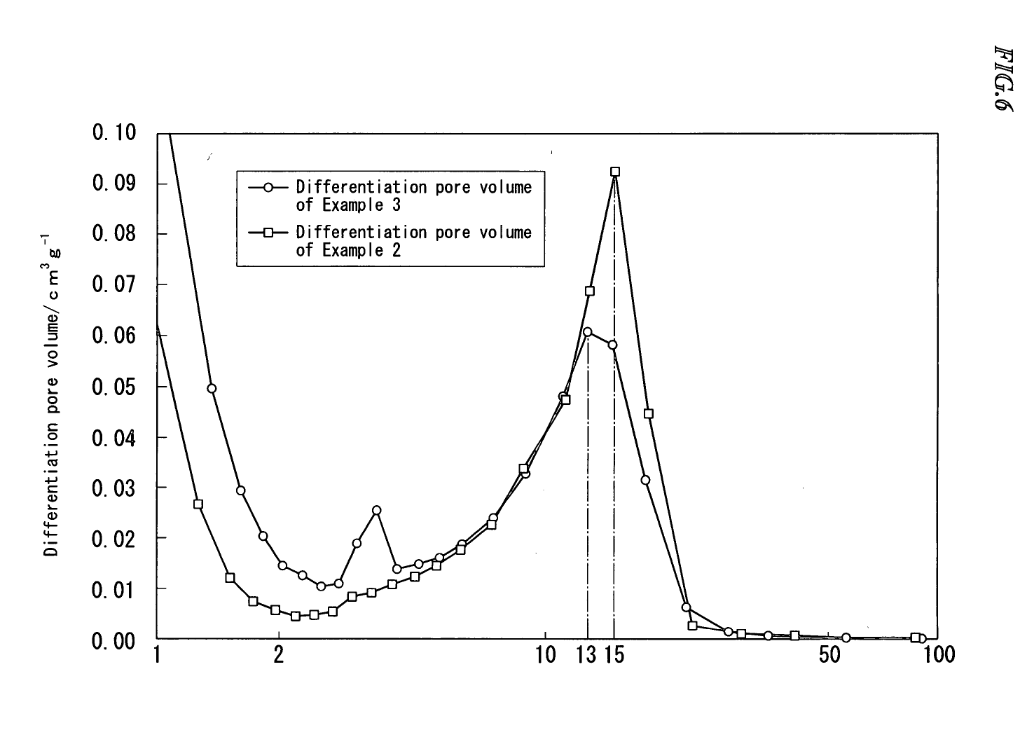

Next, the correlation between the pore diameter and the differentiation value of accumulated pore volume is shown in FIG. 5. The differentiation value of the accumulated pore volume in FIG. 5 is obtained by differentiating the accumulated pore volume by the pore diameter. It should be noted that the dotted line in FIG. 5 denotes the accumulated pore volume value of Examples 2 and 3. As shown in FIG. 5, in the differentiation pore volume of Example 3, the pore diameter is higher than that of Example 2 in the range of 2.1 μm to 25 μm. Therefore, it turns out that as to a through pore having a diameter of ranging from 2.1 μm to 25 μm, the Example 3 is more than Example 2. Although it will be clear by the liquid-passing-through experiment shown below, thereby it turns out that the analytical pretreatment column using the inorganic type filler of Example 3 excels in the liquid-passing-through property. On the other hand, in Example 2, although the differentiation pore volume value is high in the area of ranging from about 0.38 μm to less than 2.1 μm, it is thought that this phenomenon contributes a little to the liquid-passing-through property when filling up the column with this filler, because this pore volume diameter itself is present in a small area.

Furthermore, FIG. 6 denotes a graph which shows the correlation between the pore diameter and the differentiation value of accumulated pore volume, in which the pore diameter shown as a horizontal axis is expanded in the area ranging from 1 nm to 100 nm. As shown in FIG. 6, it turns out that each of differentiation curves of Examples 2 and 3 shows a peak shape at an area ranging from 2 nm to 50 nm. Therefore, it turns out that as to Examples 2 and 3, the meso pores having a diameter of 2 nm to 50 nm exists to be sure.

“The Adding and Recovery Test 1 of an Index Compound”

Next, the adding and recovery test of an index compound was performed using the analytical pretreatment column of Examples 1 and 2 and Comparative Example 11

First, as an index compound, a phenol, a cresol, and an asulam which was a kind of a disinfectant, which are considered to have a high hydrophilic property and were thought to be difficult to be recovered, were selected, respectively, and a 10 g/L solution thereof was prepared, and considered to be a standard solution. 20 μL of this standard solution was extracted by a micro syringe, added into water, and pH thereof was adjusted to 3.5 with 6N hydrochloric acid (HCl) to obtain a test water of 1 L.

A 100 ml of thus obtained test water was poured into the analytical pretreatment column of Examples 1 and 2 and Comparative Example 1, and the test water was flowed down. It should be noted that the conditions of the linear rate when flowing down the test water were set to be three conditions of 8.8 cm/minute, 22.1 cm/minute, and 35.4 cm/minute. Next, as an eluant, a 3 ml of methanol was flowed down to each column, and the effluents were collected in a round bottom flask. As to the collected effluents, a quantitative analysis was performed by the HPLC method to determine the index compounds contained in the effluent to calculate the amount of recoveries, thereby obtaining the adding and recovering percentage of the index compounds from this result.

The adding and recovering percentage of the index compound was shown in Table 2 for each condition of linear rate.

It should be noted that the adding and recovering percentage of the index compound is the mass percentage of the index compound content (μg) in each of the effluents of linear velocity of 22.1 cm/minute, and of 35.4 cm/minute, to the index compounds content (μg) in the effluent which was obtained by being flowed down at the linear rate of 8.8 cm/minute.

TABLE 2

| | Linear rate: 8.8 cm/min. | | | Linear rate: 22.1 cm/min. | | | Linear rate: 35.4 cm/min. | | |
|---|---|---|---|---|---|---|---|---|---|
| | Phenol | Cresol | Asulam | Phenol | Cresol | Asulam | Phenol | Cresol | Asulam |
| Example 1 | 100 | 103 | 100 | 101 | 96 | 97 | 97 | 94 | 92 |
| Example 2 | 100 | 99 | 100 | 89 | 90 | 89 | 66 | 75 | 59 |
| Example 3 | 100 | 101 | 100 | 75 | 82 | 69 | 60 | 70 | 42 |

(The values in the table represent the adding and recovering percentage: %)

As shown in Table 2, it turns out that in Example 1, even when the linear rate was increased by about 4 times from 8.8 cm/minute to 35.4 cm/minute, the recovery percentage scarcely deteriorated, but the index compound is fully held even if linear rate is large. That is, a quick pretreatment is possible.

In addition, it turns out that in Example 2 using a powdery filler, although a recovery percentage fell a little compared to Example 1 using a rod-like filler, even if liquid-passing speed becomes large compared to the result of the particle-like silica of Comparative Example 1, the index compound can fully be held.

In Comparative Example 1, the recovery percentage fell sharply as the linear rate increases. That is, it turns out that it becomes hard to hold the index compound as the linear rate increases, such that a part of the index compound was flowed down from the analytical pretreatment column before the eluant was flowed. In Comparative Example 1, linear rate cannot increase sharply, and hence it seems that a quick pretreatment is impossible.

“The Adding and Recovering Test 2 of an Index Compound”

The adding and recovering test of an index compound was performed using the analytical pretreatment column of Example 3 and Comparative Examples 1 and 2.

First, as an index compound, a 17β-estradiol, which is one kind of an endocrine disrupter, was selected, and a 0.01 g/L solution thereof was prepared, and considered it as standard solution. 10 μL of this standard solution was extracted by a micro syringe, added into water, and pH thereof was adjusted to 3.5 with 6N hydrochloric acid (HCl) to obtain a test water of 1 L.

1000 ml of thus obtained test water was poured into the analytical pretreatment column of Example 3 and Comparative Examples 1 and 2, and the test water was flowed down. It should be noted that the conditions of the linear rate when flowing down the test water were set to be four conditions of 17.7 cm/minute, 35.4 cm/minute, 53.1 cm/minute, and 70.8 cm/minute. After washing with a 5 ml of pure water and a 5 ml of hexane, subsequently, as an eluant, a 5 ml of an ethyl acetate/a methanol (volume ratio of 5/1) was flowed down to each column, and the effluents were collected in an eggplant flask. As to the collected effluents, a quantitative analysis was performed by the HPLC method to determine the index compounds contained in the effluent to calculate the amount of recoveries, thereby obtaining the adding and recovering percentage of the index compounds from this result.

The adding and recovering percentage of the index compound is shown in Table 3 for each condition of linear rate. It should be noted that the adding and recovering percentage of the index compound is the mass percentage of the index compound content (μg) to the index compounds content (μg) in the effluent.

TABLE 3

| | Linear rate: | | Linear rate: | | Linear rate: | | Linear rate: | |
|---|---|---|---|---|---|---|---|---|
| | 17.7 cm/min | | 35.4 cm/min | | 53.1 cm/min | | 70.8 cm/min | |
| | 17β- | | 17β- | | 17β- | | 17β- | |
| | estradiol | Estrone | estradiol | Estrone | estradiol | Estrone | estradiol | Estrone |
| W | 101 | 139 | 98 | 79 | 105 | 76 | 96 | 74 |
| 3 | | | | | | | | |
| C1 | 101 | 81 | 98 | 89 | 103 | 89 | 79 | 74 |
| C2 | 20 | 44 | 19 | 39 | 19 | 27 | 19 | 37 |

W3: Example 3, C1: Comparative Example 1, C2: Comparative Example 1
(The values in the table represent the adding and recovering percentage: %)

As shown in Table 2, it turns out that in Example 3 and Comparative Example 1, even when the linear rate was increased by about 4 times from 17.7 cm/minute to 70.8 cm/minute, the recovery percentage scarcely deteriorated, but the index compound is fully held even if linear rate is large. That is, a quick pretreatment is possible.

On the other hand, it turns out that in Comparative Example 2, the recovery rate fell although a filler having the same particle diameter as in Example 3 which has double pore structure, and that the index compound cannot fully be held. This shows that some index compounds have flown out of the analytical pretreatment column, before flowing the eluant, and it seems that the filler does not have a sufficient holding capability required for the pretreatment.

“A Liquid-Passing Through Test”

Using the cartridge prepared in Example 3, Comparative example 1, and Comparative Example 2, a liquid-passing through test was performed under the following conditions.

Conditions

As an examination water, a 1000 ml of water which contains SS (suspended particulate matter) in an amount of 8 mg/L., TC (total carbon) in an amount of 17.3 mg/L., and TOC (total organic carbon) in an amount of 5.2 mg/L. was passed through each cartridge at a fixed suction pressure (300 hPa) and the change of the flow rate was recorded at every total flowing amount.

In Table 4, liquid-passing-through rates at a certain liquid-passing-through amount are shown.

As shown in Table 4, at any cases, as the liquid-passing-through amount increases, the liquid-passing-through rate decreases, and hence it turns out that the cartridge has become plugged. In particular, as for the cartridge of Comparative Example 1, which has an adding and recovering percentage being equivalent to that of Example 3 as shown in Table 3, the plugging is remarkable, and hence it turns out that it is difficult to pass the examination water in an amount of 1000 ml through the cartridge using this suction pressure.

TABLE 4

| | Liquid-passing-through amount (ml) | | | | | | | | | |
|---|---|---|---|---|---|---|---|---|---|---|
| | 100 ml | 200 ml | 300 ml | 400 ml | 500 ml | 600 ml | 700 ml | 800 ml | 900 ml | 100 ml |
| Example 3 | 120 | 86 | 71 | 61 | 56 | 49 | 41 | 38 | 37 | 31 |
| Comparative | 54 | 44 | 32 | 25 | 17 | 11 | 5 | 1 | Almost | Almost |
| Example 1 | | | | | | | | | 0 | 0 |
| Comparative | 150 | 130 | 120 | 111 | 103 | 103 | 91 | 77 | 68 | 63 |
| Example 2 | | | | | | | | | | |

(The values in the table represent the liquid-passing-through rate: ml/min)

The above results demonstrate that what can attain both a high holding capability and a high liquid-passing-through property is only the cartridge shown in Example 3, this is because the filler filled in the cartridge shown in Example 3 has the double pore structure of the present invention.

### INDUSTRIAL APPLICABILITY

The present invention is applicable to an analytical pretreatment column for dispensing organic substances contained by a very small amount in the environment such as in water and in the atmosphere, to the process for producing an inorganic type filler for the analytical pretreatment column, to a process for dispensing a target substance, and to an environmental purification technology which uses the above, alternatively to a recovery technology for trace ingredients in very small amounts.

1. An analytical pretreatment column characterized by being filled with at least an inorganic type filler comprising a double pore structure having both a through pore with a number average diameter of not less than 0.5 micrometers to not higher than 25 micrometers, and a meso pore with a number average diameter of not less than 2 nm to not higher than 50 nm.

2. An analytical pretreatment column as set forth in claim 1, wherein the percentage of the pore volume of the through pore which occupies the volume of the inorganic type filler is ranging from not less than 30% to not higher than 90%.

3. An analytical pretreatment column as set forth in claim 1, wherein the inorganic type filler has a coating of an organic compound thereon.

4. An analytical pretreatment column as set forth in claim 1, wherein the inorganic type filler consists of a silica as a main component.

5. An analytical pretreatment column as set forth in claim 1, wherein the inorganic type filler has a granular shape, a number average diameter of not less than 10 micrometers to not more than 1000 micrometers, and the specific surface area according to the BET method of not less than 50 m²/g.

6. A process for producing an inorganic type filler, comprising:
(1)hydrolyzing and gelating alkoxysilane in a solution containing a template compound to form a silica gel,
(2) grinding the silica gel, and then treating the silica gel with a basic solution, or, treating the silica gel with a basic solution, and then grinding the silica gel, and,
(3) calcining the silica gel.

7. A process for producing an inorganic type filler as set forth in claim 6 wherein the grinding the silica gel is performed by using a device having a porous plate with plural penetrating holes which squeezes the silica gel into the penetration holes from one surface of the plate to grind the silica gel.

8. A process for producing an inorganic type filler as set forth in claim 6, wherein the template compound is any one of polyethylene oxide, poly oxy ethylene alkyl ether, and poly acrylic acid.

9. An analytical pretreatment column as set forth in claim 1, wherein the inorganic type filler is one produced by a process for producing an inorganic type filler, comprising:
(1) hydrolyzing and gelatins alkoxysilane in a solution containing a template compound to form a silica eel,
(2) grinding the silica gel, and then treating the silica gel with a basic solution, or, treating the silica gel with a basic solution, and then grinding the silica gel, and,
(3) calcining the silica gel.

10. A process for dispensing a dispense target substance, comprising:
flowing an analyzed sample containing a dispense target substance and a coexisting substance into the analytical pretreatment column as set forth in claim 1, thereby making the dispense target substance be adsorbed to the inorganic type filler, and
flowing an eluting solvent into the analytical pretreatment column, thereby debouching the dispense target substance from the analytical pretreatment column.

11. A process for dispensing a dispense target substance, comprising:
flowing an analyzed sample containing a dispense target substance and a coexisting substance into the analytical pretreatment column as set forth in claim 1, thereby making the dispense target substance be adsorbed to the inorganic type filler and debouching the dispense target substance from the analytical pretreatment column.

12. A process for dispensing a dispense target substance as set forth in claim 10, wherein the dispense target substance is one or more selected from the group consisting of dioxin, polychlorinated biphenyl, agricultural chemicals, endocrine disrupter, heavy metal, and protein.

13. A process for purifying a sample, comprising:
flowing a sample containing a removal target substance and a coexisting substance into the analytical pretreatment column as set forth in claim 1, thereby removing the removal target substance from the sample.

14. A process for recovering a recovery target substance, comprising:
flowing a sample containing a recovery target substance and a coexisting substance into the analytical pretreatment column as set forth in claim 1, thereby recovering the recovery target substance from the sample.

15. An analytical pretreatment column as set forth in claim 9, wherein the grinding the silica gel is performed by using a device having a porous plate with plural penetrating holes which squeezes the silica gel into the penetration holes from one surface of the plate to grind the silica gel.

16. An analytical pretreatment column as set forth in claim 9, wherein the template compound is any one of polyethylene oxide, poly oxy ethylene alkyl ether, and poly acrylic acid.

17. A process for dispensing a dispense target substance as set forth in claim 11, wherein the dispense target substance is one or more selected from the group consisting of dioxin, polychlorinated biphenyl, agricultural chemicals, endocrine disruptor, heavy metal, and protein.
